(19) United States
(12) Patent Application Publication
(10) Pub. No.: US 2020/0170993 A1
(43) Pub. Date: Jun. 4, 2020

(54) LIPOSOMAL PACLITAXEL FORMULATION FOR TREATING BLADDER CANCER

(71) Applicant: TESORX PHARMA, LLC, Menlo Park, CA (US)

(72) Inventors: Michael G. OEFELEIN, Tustin, CA (US); Guru V. BETAGERI, Chino Hills, CA (US); Natarajan VENKATESAN, Diamond Bar, CA (US)

(21) Appl. No.: 16/631,566

(22) PCT Filed: Jul. 19, 2018
(86) PCT No.: PCT/US2018/042857

§ 371 Date: Jan. 16, 2020

Related U.S. Application Data
(60) Provisional application No. 62/534,285, filed on Jul. 19, 2017.

(51) Int. Cl. A61K 31/337; A61K 47/24; A61K 47/28; A61K 47/26; A61K 47/18; A61K 33/243; A61K 9/00

(52) U.S. Cl. CPC A61K 31/337; A61K 47/24; A61K 47/28; A61K 9/0034; A61K 47/183; A61K 33/243; A61K 47/26

(57) ABSTRACT

Compositions and methods for making and using liposomal formulations of paclitaxel are disclosed. The liposomal paclitaxel formulations are used with treatment regimens for bladder cancer and both lower and upper tract urothelial cancer. Hence, the formulations are suitable to treat bladder cancers by intravesical administration and to treat urothelial cancers. The formulations according to the invention include paclitaxel, lecithin, cholesterol, threonine, and glucose.

### CROSS-REFERENCE TO RELATED APPLICATIONS

This application claims the benefit of U.S. Provisional Application No. 62/534,285, filed Jul. 19, 2017, which is incorporated herein by reference.

### FIELD OF THE INVENTION

The inventions described herein relate to the administration of liposome formulations comprising paclitaxel, lecithin, cholesterol, threonine, and glucose, for use in the treatment of bladder cancer and upper urinary tract carcinomas.

### SUMMARY OF THE INVENTION

The invention relates to a use of a liposome formulation comprising paclitaxel, lecithin, cholesterol, threonine, and glucose (“a liposome formulation of the invention”) to treat bladder cancer or upper tract urothelial carcinoma (UTUC) in an individual in need thereof. Liposome formulations of the invention are typically administered by intravesical delivery to the bladder; however, suitable retrograde or antegrade methods for delivering liposome formulations to the upper urinary tract and renal pelvis are also acceptable in methods of the invention.

Liposome formulations of the invention are administered as suspensions in an aqueous solution, such as saline solution or water. The administered liposome formulations of the invention treat bladder cancer, a general term that is inclusive of bladder, ureter, renal pelvis and urothelial renal carcinomas. Upper tract urothelial carcinomas occur in the upper urinary tract, including the ureter and renal pelvis of the kidney. Liposome formulations administered by methods of the invention may also include one or more externally-added pharmaceutically acceptable excipients. A liposome formulation of the invention may also be administered as part of a co-therapy which further includes at least one therapeutic drug in addition to paclitaxel.

### DETAILED DESCRIPTION OF THE INVENTION

The invention relates to methods of using liposome formulations which contain paclitaxel, lecithin, cholesterol, threonine, and glucose (“liposome formulations of the invention”) to treat bladder cancer or upper tract urothelial carcinoma. A commercially available embodiment of a liposome formulation that is used in methods of the invention is marketed as Lipusu®.

A method of use according to the invention, typically administers a therapeutic dose of a liposome formulation into the bladder via intravesical delivery. Intravesical therapy involves instillation of a therapeutic agent directly into the bladder via insertion of a urethral catheter. In a typical protocol of an intravesical instillation, sterile catheterization can be performed with a straight or a coude (male) catheter. The bladder is emptied completely. A catheter tip syringe can be inserted containing the treatment with an adaptor at the tip of the syringe to prevent spillage or splash during insertion. Alternatively, primed tubing attached to medication vial can be inserted into catheter and a chemotherapeutic agent is instilled per gravity flow or by gentle injection. The syringe or medication vial used in the catheter system can be removed with tubing intact. The catheter is squeezed closed, and then plugged or removed, using sterile gauze to help absorb any drops. If the patient receiving the administered liposome formulation has trouble holding the solution in the bladder, a Foley catheter may be used and a catheter plug may be inserted onto the end of the catheter after instillation so that liposome formulation remains in the bladder for a specified amount of time, usually one to two hours, but less if a shorter period of time is therapeutically effective. A patient may be instructed to lie down and reposition periodically to dislodge air bubbles from catheter and to insure medication comes in contact with all areas of the bladder. Depending on patient's mobility, the catheter may be removed at the end of the desired dwell time or patient may be connected to a urinary drainage bag to drain the chemotherapeutic agent. Examples of intravesical drug delivery devices and methods for deploying those devices into the bladder are described in the following U.S. Patent Application Publications: U.S. 2015/0165178; U.S. 2012/0203203; U.S. 2012/0089122; U.S. 2012/0089121; U.S. 2011/0218488; U.S. 2011/0202036; U.S. 2011/0152839; U.S. 2011/0060309; U.S. 2010/0331770; U.S. 2010/0330149; U.S. 2010/0003297; U.S. 2009/0149833; and U.S. 2007/0202151, which are all incorporated herein in their entireties.

Other methods of use according to the invention, administer a therapeutic dose of a liposome formulation into the upper urinary tract, including the renal pelvis of the kidney. Various methods are available for delivering a liposome formulation to the upper urinary tract, including stent-type devices with a balloon portion, which resides within the bladder and is filled with a liquid suspension of a liposome formulation of the invention after the device is deployed in the bladder

In addition to intravesical delivery, the formulations and dosage forms of the invention can be administered into the ureter and/or renal pelvis using an appropriate ureteral access catheter or nephrostomy catheter device and protocol known in the art. Such delivery of a chemotherapeutic agent can be used to treat, for example, upper tract urothelial carcinoma.

Generally, irrespective of whether a method of the invention delivers the liposome formulation to the bladder, upper urinary tract or renal pelvis, the liposome formulation is suspended in an aqueous solution prior to administration. For example, a liposome formulation may be suspended in water, preferably sterile water or sterile distilled water. Alternatively, a liposome formulation may be suspended in a sterile saline solution, preferably a 0.9% NaCl saline solution. Pharmaceutically acceptable buffered solutions, such as phosphate buffered saline (PBS) are also acceptable for suspending liposome formulations in methods of the invention.

As stated above, a method of the invention administers a liposome formulation to treat bladder cancer or upper tract urothelial carcinoma (UTUC). Indeed, various types of renal neoplasms and urothelial carcinomas are treated by methods of the invention, including non-muscle invasive bladder cancer (NMIBC), squamous cell carcinoma, adenocarcinoma and urothelial carcinoma, which is also called transitional cell carcinoma. Urothelial carcinoma is the most common type of bladder cancer, accounting for about 90 percent of bladder cancer all cases. These cancers are usually superficial in about 75 percent of cases, where they have not advanced into the deeper layers of the bladder wall. The term upper tract urothelial carcinoma generally refers to renal neoplasms and urothelial carcinomas that occur in the upper urinary tract.

A therapeutic dosage amount of a liposome formulation of the invention generally contains an amount of paclitaxel sufficient to remove all symptoms or at least partially alleviate at least one of the symptoms of bladder cancer or UTUC for an individual experiencing bladder cancer or UTUC. A particular therapeutically effective dosage amount depends on the stage, severity and course of the bladder cancer, previous therapy, the individual's health status, weight, response to the drugs, and/or the judgment of the treating physician. Exemplary therapeutic dosages for a method of the invention include an amount of paclitaxel that ranges from is 1 to 1000 mg/m². For example, the therapeutic dosage may be from: 1 to 100 mg/m²; 50 to 150 mg/m²; 100 to 200 mg/m²; 150 to 250 mg/m²; 200 to 300 mg/m²; 350 to 450 mg/m²; 400 to 500 mg/m²; 450 to 550 mg/m²; 500 to 600 mg/m²; 550 to 650 mg/m²; 600 to 700 mg/m²; 750 to 850 mg/m²; 800 to 900 mg/m²; 850 to 950 mg/m²; 900 to 1000 mg/m²; or 9500-1000 mg/m².

A method of the invention may also administer a liposome formulation to which at least one pharmaceutically suitable excipient has been added. For example, a lyophillized liposome formulation according to the invention may be admixed with at least one pharmaceutically acceptable excipient. Exemplary pharmaceutically acceptable excipients include, but are not limited to: (a) cryoprotectant, fillers, or extenders, such as, for example, mannitol, starches, lactose (e.g., lactose monohydrate), sucrose, glucose, trehalose, and silicic acid; (b) binders, such as, for example, cellulose derivatives, including hydroxypropyl methyl cellulose, which is available commercially as Benecel™, hydroxypropyl cellulose, which is available commercially as Klucel™ (Ashland Inc Covington, Ky.), starch, aliginates, gelatin, polyvinylpyrrolidone, sucrose, and gum acacia, (c) absorption accelerators, such as, for example, quaternary ammonium compounds.

In certain instances, it is appropriate for a method of the invention to administer another therapeutic agent as part of a co-therapy for treating bladder cancer or UTUC. For example, in certain co-therapies for treating bladder cancer or UTUC, 5-fluorouracil (5-FU), or cisplatin is administered in addition to a liposome formulation of the invention. Where combinational therapy is employed, other agents do not have to be administered in the same pharmaceutical composition as the liposome formulation, and can be, because of different physical and chemical characteristics, administered by different routes. An additional therapeutic agent can be administered concurrently (e.g., simultaneously, essentially simultaneously, or within the same treatment protocol) or sequentially, depending upon the stage and type of bladder cancer or UTUC, the condition of the patient, and the actual choice of compounds used. The determination of the order of administration, and the number of repetitions of administration of each therapeutic agent during a treatment protocol, can be based upon evaluation of the disease being treated and the condition of the individual.

1. Use of a liposome formulation comprising paclitaxel, lecithin, cholesterol, threonine, and glucose to treat bladder cancer or upper tract urothelial carcinoma in an individual in need thereof.

2. The use of claim 1, wherein the liposome formulation is administered by intravesical delivery.

3. The use of claim 2, wherein the liposome formulation is suspended in an aqueous solution prior to administration.

4. The use of claim 3, wherein the aqueous solution is saline solution or water.

5. The use of claim 2, wherein the liposome formulation treats bladder cancer.

6. The use of claim 5, wherein the bladder cancer is a non-muscle invasive bladder cancer.

7. The use of claim 1, wherein the liposome formulation is administered into the ureter or renal pelvis or both.

8. The use of claim 7, wherein the liposome formulation treats upper tract urothelial carcinoma.

9. The use of claim 8, wherein the liposome formulation is suspended in an aqueous solution prior to administration.

10. The use of claim 9, wherein the aqueous solution is saline solution or water.

11. The use of claim 1, wherein the dose of the paclitaxel is 135 to 175 mg/m².

12. The use of claim 1, wherein the liposome formulation further comprises at least one pharmaceutically acceptable excipient.

13. The use of claim 12, wherein the dosage of the paclitaxel is 1 to 1000 mg/m².

14. The use of claim 1, wherein the liposome formulation is administered as part of a co-therapy comprising least one therapeutic drug in addition to paclitaxel.

15. The use of claim 14, wherein the at least one additional chemotherapeutic drug is cisplatin.

16. The use of claim 14, wherein the liposome formulation further comprises at least one pharmaceutically acceptable excipient.

17. The use of claim 14, wherein the dose of the paclitaxel is 1 to 1000 mg/m².
